(19) United States
(12) Patent Application Publication
(10) Pub. No.: US 2026/0096877 A1
(43) Pub. Date: Apr. 9, 2026

(54) APPARATUS AND METHODS FOR 3D PRINTING INTRAOCULAR LENS COMPONENTS, INTRAOCULAR LENS FORMULATIONS SUITABLE FOR 3D PRINTING, AND 3D-PRINTED INTRAOCULAR LENS COMPONENTS

(71) Applicant: Alcon Inc., Fribourg (CH)

(72) Inventors: Douglas Charles SCHLUETER, Azle, TX (US); Shridhar NATARAJAN, Keller, TX (US); Mihir M. KALE, Coppell, TX (US); John JANDERS, Fort Worth, TX (US)

(73) Assignee: Alcon Inc., Fribourg (CH)

(21) Appl. No.: 19/326,387

(22) Filed: Sep. 11, 2025

Related U.S. Application Data
(60) Provisional application No. 63/703,001, filed on Oct. 3, 2024.

(51) Int. Cl. A61F 2/16; A61L 27/16; B33Y 10/00; B33Y 30/00; B33Y 70/10; B33Y 80/00; C09D 4/00; C09D 133/04; G02B 1/04

(52) U.S. Cl. CPC A61F 2/16; A61L 27/16; B33Y 10/00; B33Y 30/00; B33Y 70/10; B33Y 80/00; C09D 4/00; C09D 133/04; G02B 1/041; A61F 2002/1686; A61F 2002/169; A61F 2002/16902; A61F 2240/001

(57) ABSTRACT

Disclosed are apparatus and methods for 3D printing intraocular lens components, intraocular lens formulations suitable for 3D printing, and 3D-printed intraocular lens components. In one aspect, the intraocular lens formulation can comprise a plurality of monomers, a crosslinkable polymer comprising the plurality of monomers, a crosslinker, and a photoinitiator. Also disclosed is a 3D printer for printing an intraocular lens component. The 3D printer can comprise a reservoir configured to contain an intraocular lens formulation, a build platform comprising a build surface configured to be initially in fluid contact with the intraocular lens formulation within the reservoir, a light source configured to generate a light, and at least one of a mirror and a projection optic configured to direct the light generated by the light source at the intraocular lens formulation within the reservoir to cure and form one layer of the intraocular lens component on the build surface.

### CROSS-REFERENCE TO RELATED APPLICATION

This application claims the benefit of priority to U.S. Provisional Patent Application No. 63/703,001 filed on Oct. 3, 2024, the content of which is incorporated herein by reference in its entirety.

### TECHNICAL FIELD

The present disclosure relates generally to the field of intraocular lenses, and, more specifically, to apparatus and methods for 3D printing intraocular lens components, intraocular lens formulations suitable for 3D printing, and 3D-printed intraocular lens components.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the crystalline lens matrix from the patient's lens capsule and replaces it with an intraocular lens (IOL).

However, the manufacturing process for certain newer IOLs can be complicated and require many different types of equipment and procedural steps. In some cases, the geometries of certain intraocular lens components can be constrained by these different types of equipment and procedural steps. This is especially true for IOL components with intricate designs and geometries.

Therefore, a solution is needed which can reduce the amount of equipment needed to produce certain IOL components. Such a solution should also not be overly complicated and should be cost-effective.

### SUMMARY

Disclosed herein are apparatus and methods for 3D printing intraocular lens components and intraocular lens formulations suitable for 3D printing. In some embodiments, an intraocular lens formulation suitable for 3D printing is disclosed. The intraocular lens formulation can comprise a plurality of monomers, a crosslinkable polymer comprising the plurality of monomers, a crosslinker, and a photoinitiator.

The plurality of monomers can comprise an alkyl acrylate and/or alkyl methacrylate, and a phenyl acrylate or phenyl methacrylate. The plurality of monomers can also comprise a fluoromethacrylate or a fluoroacrylate. For example, the plurality of monomers can comprise an alkyl acrylate and/or alkyl methacrylate, a phenyl acrylate or phenyl methacrylate, and a fluoromethacrylate or a fluoroacrylate.

In some embodiments, the alkyl acrylate can be butyl acrylate or the alkyl methacrylate can be butyl methacrylate. The alkyl acrylate or the alkyl methacrylate can be between 10% and 30% of the intraocular lens formulation (wt %).

In some embodiments, the phenyl acrylate can be phenylethyl acrylate or the phenyl methacrylate can be phenylethyl methacrylate. The phenyl acrylate or the phenyl methacrylate can be between 30% and 60% of the intraocular lens formulation (wt %).

In some embodiments, the fluoromethacrylate can be a trifluoroethyl methacrylate or the fluoroacrylate can be a trifluoroethyl acrylate. When the plurality of monomers comprise the fluoromethacrylate or the fluoroacrylate, the fluoromethacrylate or the fluoroacrylate can be between 10% and 20% of the intraocular lens formulation (wt %).

In some embodiments, the crosslinker can be ethylene glycol dimethacrylate (EGDMA). The crosslinker can be between 0.1% and 5.0% of the intraocular lens formulation (wt %).

In some embodiments, the crosslinkable polymer can be between about 5% and 40% of the intraocular lens formulation (wt %). In certain embodiments, the crosslinkable polymer can be less than 40% of the intraocular lens formulation (wt %).

In some embodiments, the crosslinkable polymer can comprise the alkyl acrylate and/or the alkyl methacrylate, the phenyl acrylate or the phenyl methacrylate, a monomer comprising a hydroxyl moiety, a curing agent, and, optionally, the fluoromethacrylate or the fluoroacrylate. In certain embodiments, the alkyl acrylate or the alkyl methacrylate can be between 40% and 45% of the crosslinkable polymer (wt %), the phenyl acrylate or the phenyl methacrylate can be between 25% and 35% of the crosslinkable polymer (wt %), and the fluoromethacrylate or the fluoroacrylate can be between 20% and 25% of the crosslinkable polymer (wt %).

In some embodiments, the monomer comprising the hydroxyl moiety can be hydroxyethyl acrylate (HEA).

In some embodiments, the curing agent can be an alpha-hydroxyketone.

In some embodiments, the photoinitiator can be between 0.1% and 5% of the intraocular lens formulation (wt %). For example, the photoinitiator can be bis(2,4,6-trimethylbenzoyl)-phenylphosphineoxide.

In some embodiments, the plurality of monomers can be passed through a column of basic alumina prior to being added to the intraocular lens formulation. The crosslinker can also be passed through a column of basic alumina prior to being added to the intraocular lens formulation.

In some embodiments, the intraocular lens formulation can be curable by ultraviolet (UV) light. A wavelength of the UV light can be between about 365 nm and about 410 nm. In some embodiments, the intraocular lens formulation can be in liquid form prior to being cured by light energy.

In some embodiments, a method of 3D printing an intraocular lens component is disclosed. The method can comprise: (i) introducing an intraocular lens formulation into a reservoir of a 3D printer, (ii) directing light generated by a light source of the 3D printer to a portion of the intraocular lens formulation within the reservoir to cure the portion of the intraocular lens formulation and form one layer of the intraocular lens component on a build surface of the 3D printer, (iii) translating at least one of the build surface and the reservoir in a z-direction after the one layer of the intraocular lens component is formed, and (iv) repeating steps (ii) and (iii) until all layers of the intraocular lens component are formed.

The method can further comprise passing monomers of the intraocular lens formulation through a column of basic alumina and introducing the intraocular lens formulation comprising the monomers having passed through the column of basic alumina into the reservoir of the 3D printer. In some embodiments, the monomers are passed through the column of basic alumina without a solvent.

In some embodiments, the light generated by the light source can be ultraviolet (UV) light. For example, a wavelength of the UV light can be between 365 nm and 410 nm.

In some embodiments, an exposure time of the intraocular lens formulation to the light can be between 0.1 seconds and 10.0 seconds.

The method can further comprise waiting between 1 second and 900 seconds in between light exposures.

The method can further comprise coupling a glass plate to the build surface and forming the layer of the intraocular lens component on the glass plate.

The method can also comprise rinsing the intraocular lens component with isopropyl alcohol after all layers of the intraocular lens component are formed.

The method can further comprise post-curing the intraocular lens component after the intraocular lens component is rinsed with the isopropyl alcohol. In some embodiments, the intraocular lens component can be post-cured using UV light, heat, or a combination thereof. For example, the intraocular lens component can be post-cured for at least 30 minutes.

In some embodiments, the 3D printer can be a digital light processing (DLP) 3D printer, a projection micro-stereolithography 3D printer, another type of stereolithography 3D printer, or a two photon polymerization (2PP) 3D printer.

In some embodiments, the 3D printer can have a print resolution of between 2 μm and 30 μm.

In some embodiments, each layer of the intraocular lens component can have a thickness between 5 μm and 50 μm.

In some embodiments, the intraocular lens component can be a haptic of an intraocular lens.

In some embodiments, a 3D printer for printing an intraocular lens component is disclosed. The 3D printer can comprise a reservoir configured to contain an intraocular lens formulation, a build platform comprising a build surface, a light source configured to generate a light, and at least one of a mirror and a projection optic configured to direct the light generated by the light source at the intraocular lens formulation within the reservoir to cure a portion of the intraocular lens formulation and form one layer of the intraocular lens component on the build surface.

In some embodiments, the build surface can be configured to be initially in fluid contact with the intraocular lens formulation within the reservoir. At least one of the reservoir and the build platform can be translatable in a z-direction.

In some embodiments, the 3D printer can further comprise one or more actuators configured to translate at least one of the reservoir and the build platform in a z-direction after each layer of the intraocular lens component is formed on the build surface.

In some embodiments, the light generated by the light source of the 3D printer can be ultraviolet (UV) light. For example, a wavelength of the UV light can be between 365 nm and 410 nm.

In some embodiments, the 3D printer can further comprise a glass plate coupled to the build surface. At least one layer of the intraocular lens component can be formed on the glass plate.

Also disclosed are 3D-printed intraocular lens components such as 3D-printed haptics. In some embodiments, a 3D-printed haptic can comprise a 3D-printed haptic body comprising a radially-outer haptic surface and a plurality of 3D-printing support structure remnants protruding from the radially-outer haptic surface. The 3D-printing support structure remnants can be formed by removing portions of 3D-printing support structures used to support a part of the 3D-printed haptic during a 3D printing process.

Also disclosed is a method of 3D printing a haptic of an intraocular lens. The method can comprise 3D printing the haptic of the intraocular lens. At least part of the haptic can be supported by 3D-printing support structures during the 3D printing process. The method can also comprise removing portions of the 3D-printing support structures until 3D-printing support structure remnants remain along a surface of the haptic.

In some embodiments, 3D printing the haptic of the intraocular lens can further comprise 3D printing the haptic using a digital light processing (DLP) 3D printer.

In other embodiments, 3D printing the haptic of the intraocular lens can further comprise 3D printing the haptic using a projection micro-stereolithography 3D printer.

The haptic body of the 3D-printed haptic can have a distal free end and a proximal attachment end opposite the distal free end. The plurality of 3D-printing support structure remnants can protrude from an area of the radially-outer haptic surface proximal to the proximal attachment end.

In some embodiments, the area of the radially-outer haptic surface proximal to the proximal attachment end can be located closer to the proximal attachment end than the distal free end. The 3D-printed haptic can comprise a haptic fluid lumen extending through at least part of the 3D-printed haptic body. A haptic fluid port can be defined at the proximal attachment end. The haptic fluid port can be in fluid communication with the haptic fluid lumen.

In some embodiments, the 3D-printing support structure remnants can be shaped substantially as discrete bumps or nubs protruding from the radially-outer haptic surface. The 3D-printing support structure remnants can be made of the same material as the 3D-printed haptic body. The 3D-printing support structure remnants can be scattered along part of the radially-outer haptic surface.

A minimum height of each of the 3D-printing support structure remnants can be about 10 μm. A maximum height of each of the 3D-printing support structure remnants can be about 1000 μm or greater than 1000 μm.

In some embodiments, the haptic fluid lumen can be surrounded by a radially-outer haptic lumen wall and a radially-inner haptic lumen wall. The radially-outer haptic surface can be a radially-outer surface of the radially-outer haptic lumen wall.

In some embodiments, the step of removing the portions of the 3D-printing support structures can further comprise cutting, clipping, or trimming the 3D-printing support structures until only the 3D-printing support structure remnants remain along the surface of the haptic.

### BRIEF DESCRIPTION OF THE DRAWINGS

FIG. 1A illustrates a top plan view of one embodiment of an intraocular lens comprising one or more lens components that can be 3D printed.

FIG. 1B illustrates an exploded view of the intraocular lens of FIG. 1A.

FIG. 1C illustrates an exploded view of another embodiment of an intraocular lens comprising one or more lens components that can be 3D printed.

FIG. 2A is a schematic diagram illustrating a 3D printer for printing an intraocular lens component in operation.

FIG. 2B illustrates one embodiment of the 3D printer for printing the intraocular lens component.

FIG. 3A illustrates another embodiment of a 3D printer for printing an intraocular lens component.

FIG. 3B illustrates an intraocular lens formulation being poured into a reservoir of the 3D printer of FIG. 3A.

FIG. 3C is a schematic diagram illustrating part of the 3D printer of FIG. 3A in operation.

FIG. 4 illustrates one embodiment of a basic alumina column that can be used to filter out inhibitors from monomers of the intraocular lens formulation.

FIG. 5 illustrates one embodiment of an intraocular lens component printed on a glass plate coupled to a build surface.

FIG. 6A illustrates a top perspective view of a haptic of an intraocular lens printed using one of the 3D printers disclosed herein.

FIG. 6B illustrates a top plan view of the haptic of FIG. 6A.

FIG. 7 is a flowchart illustrating one embodiment of a method of 3D printing an intraocular lens component.

FIG. 8 is a flowchart illustrating one embodiment of a method of 3D printing an intraocular lens component.

### DETAILED DESCRIPTION

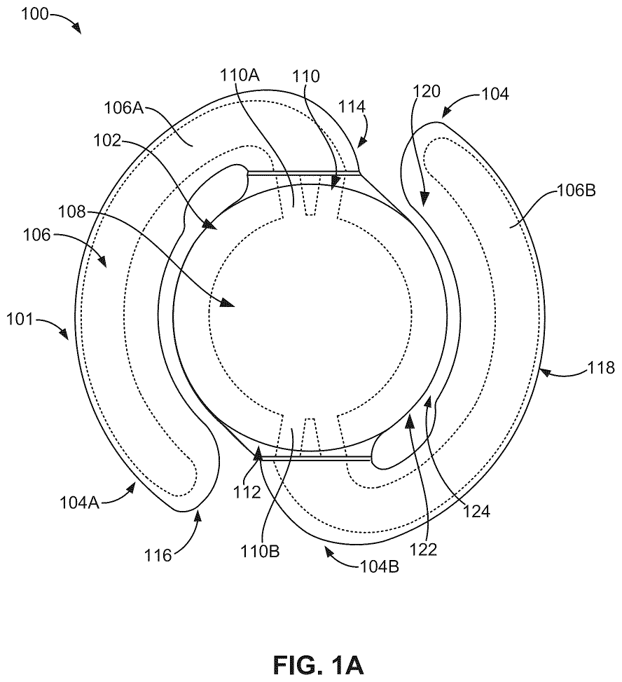

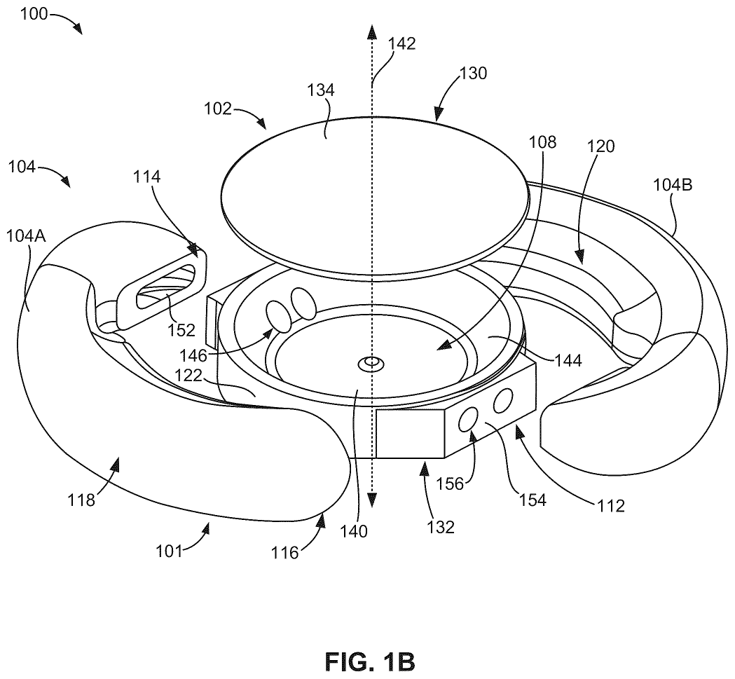

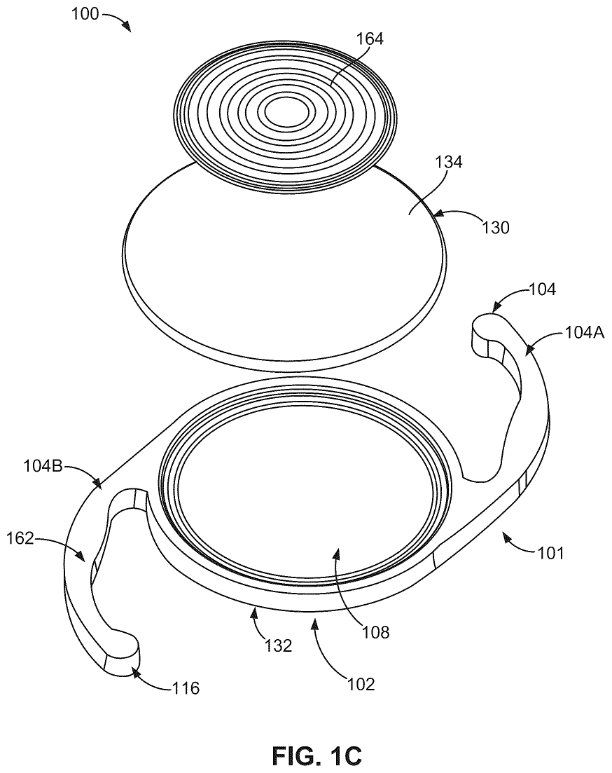

FIG. 1A illustrates a top plan view of one embodiment of an intraocular lens 100 comprising one or more lens components 101 that can be 3D printed. The intraocular lens 100 can be implanted within a subject to correct for defocus aberration, corneal astigmatism, spherical aberration, or a combination thereof.

The intraocular lens 100 can comprise an optic portion 102 and one or more haptics 104 including a first haptic 104A and a second haptic 104B coupled to and extending peripherally from the optic portion 102. For example, the intraocular lens 100 can be positioned within a native capsular bag in which a native lens has been removed. When the intraocular lens 100 is implanted within the native capsular bag, the optic portion 102 can be adapted to refract light that enters the eye onto the retina.

In some embodiments, the haptics 104 can be coupled to and adhered to the optic portion 102. For example, the haptics 104 can be adhered to the optic portion 102 after each is formed separately. In other embodiments, the intraocular lens 100 can be a one-piece lens such that the haptics 104 are connected to and extend from the optic portion 102. In this example embodiment, the haptics 104 are formed along with the optic portion 102 and are not adhered or otherwise coupled to the optic portion 102 in a subsequent step.

In some embodiments, the lens components 101 can comprise the haptics 104 and the optic portion 102. In other embodiments, the lens components 101 can comprise the one or more haptics 104.

In some embodiments, the intraocular lens 100 can be a fluid-filled IOL such as an accommodating IOL (or “AIOL”). As will be discussed in more detail in later sections, the intraocular lens 100 can also be a fluid-tunable non-accommodating intraocular lens (see, e.g., FIG. 1C).

When the intraocular lens 100 is an AIOL, one or more haptics 104 can be configured to engage the capsular bag and be adapted to deform in response to ciliary muscle movement (e.g., muscle relaxation, muscle contraction, or a combination thereof) in connection with capsular bag reshaping. Each of the haptics 104 can have a haptic body comprising a haptic fluid lumen 106 (shown in broken or phantom lines) extending through at least part of the haptic body of the haptic 104. For example, the first haptic 104A can comprise a first haptic fluid lumen 106A extending through at least part of the first haptic 104A and the second haptic 104B can comprise a second haptic fluid lumen 106B extending through at least part of the second haptic 104B. The haptic fluid lumen 106 (e.g., any of the first haptic fluid lumen 106A or the second haptic fluid lumen 106B) can be in fluid communication with or fluidly connected to an optic fluid chamber 108 within the optic portion 102.

The optic fluid chamber 108 and the haptic fluid lumen(s) 106 can comprise a fluid. A base power of the optic portion 102 can be configured to change based on an internal fluid pressure within the fluid-filled optic fluid chamber 108. The base power of the optic portion 102 can be configured to increase or decrease as fluid enters or exits the fluid-filled optic fluid chamber 108. For example, the base power of the optic portion 102 can be configured to decrease as fluid exits or is drawn out of the fluid-filled optic fluid chamber 108 into the haptic fluid lumen(s) 106. Also, for example, the base power of the optic portion 102 can be configured to increase as fluid enters the fluid-filled optic fluid chamber 108 from the haptic fluid lumen(s) 106.

The optic fluid chamber 108 can be in fluid communication with the one or more haptic fluid lumens 106 through one or more fluid channels 110. The fluid channels 110 can be conduits or passageways fluidly connecting the optic fluid chamber 108 to the haptic fluid lumens 106. The fluid channels 110 can be spaced apart from one another. For example, a pair of fluid channels 110 can be spaced apart between about 0.1 mm to about 1.0 mm. In some embodiments, each of the fluid channels 110 can have a diameter of between about 0.4 mm to about 0.6 mm.

The haptics 104 can be coupled to the optic portion 102 at a reinforced portion. The reinforced portion can serve as a haptic-optic interface 112. The pair of fluid channels 110 can be defined or formed within part of the reinforced portion.

As shown in FIG. 1A, the optic fluid chamber 108 can be in fluid communication with the first haptic fluid lumen 106A through a first pair of fluid channels 110A. The optic fluid chamber 108 can also be in fluid communication with the second haptic fluid lumen 106B through a second pair of fluid channels 110B.

In some embodiments, the first pair of fluid channels 110A and the second pair of fluid channels 110B can be positioned substantially on opposite sides of the optic portion 102. The first pair of fluid channels 110A can be positioned substantially diametrically opposed to the second pair of fluid channels 110B. The first pair of fluid channels 110A and the second pair of fluid channels 110B can extend or be defined through part of the optic portion 102. The first pair of fluid channels 110A and the second pair of fluid channels 110B can extend or be defined through a posterior element 132 of the optic portion 102 (see, e.g., FIG. 1B).

FIG. 1A also illustrates that each of the haptics 104 (e.g., any of the first haptic 104A or the second haptic 104B) can have a proximal attachment end 114 and a distal free end 116. A haptic fluid port 152 (see, e.g., FIG. 1B) can be defined at the proximal attachment end 114 of the haptic 104. The haptic fluid port 152 can serve as an opening of the haptic fluid lumen 106. Fluid within the haptic fluid lumen 106 can flow out of the haptic fluid lumen 106 through the haptic fluid port 152 and into the optic fluid chamber 108 via the fluid channels 110 when the haptic 104 is coupled to the optic portion 102. Similarly, fluid within the optic fluid chamber 108 can flow out of the optic fluid chamber 108 through the pair of fluid channels 110 and into the haptic fluid lumen 106 through the haptic fluid port 152.

Each of the haptics 104 can comprise a radially-outer haptic lumen wall 118 and a radially-inner haptic lumen wall 120. The radially-outer haptic lumen wall 118 (also referred to as a radially-outer lateral wall of the haptic 104) can be configured to face and contact an inner surface of a patient's capsular bag when the intraocular lens 100 is implanted within the capsular bag. The radially-inner haptic lumen wall 120 (also referred to as a radially-inner lateral wall of the haptic 104) can be configured to face an outer peripheral surface 122 of the optic portion 102.

As previously discussed, the intraocular lens 100 can be implanted or introduced into a patient's capsular bag after a native lens has been removed from the capsular bag. The patient's capsular bag is connected to zonule fibers which are connected to the patient's ciliary muscles. The capsular bag is elastic and ciliary muscle movements can reshape the capsular bag via the zonule fibers. For example, when the ciliary muscles relax, the zonules are stretched. This stretching pulls the capsular bag in the generally radially outward direction due to radially outward forces. This pulling of the capsular bag causes the capsular bag to elongate, creating room within the capsular bag. When the patient's native lens is present in the capsular bag, the native lens normally becomes flatter (in the anterior-to-posterior direction), which reduces the power of the lens, allowing for distance vision. In this configuration, the patient's native lens is said to be in a disaccommodated state or undergoing disaccommodation.

When the ciliary muscles contract, however, as occurs when the eye is attempting to focus on near objects, the radially inner portion of the muscles move radially inward, causing the zonules to slacken. The slack in the zonules allows the elastic capsular bag to contract and exert radially inward forces on a lens within the capsular bag. When the patient's native lens is present in the capsular bag, the native lens normally becomes more curved (e.g., the anterior part of the lens becomes more curved), which gives the lens more power, allowing the eye to focus on near objects. In this configuration, the patient's native lens is said to be in an accommodated state or undergoing accommodation.

In embodiments where the intraocular lens 100 is an AIOL, the radially-outer haptic lumen wall 118 of the implanted AIOL can directly engage with or be in physical contact with the portion of the capsular bag that is connected to the zonules or zonule fibers. Therefore, the radially-outer haptic lumen wall 118 of the AIOL can be configured to respond to capsular bag reshaping forces that are applied radially when the zonules relax and stretch as a result of ciliary muscle movements.

For example, when the ciliary muscles contract, the peripheral region of the elastic capsular bag reshapes and applies radially inward forces on the radially-outer haptic lumen wall 118 of each of the haptics 104. When the intraocular lens 100 is an AIOL, the radially-outer haptic lumen wall 118 can deform or otherwise change shape and this deformation or shape-change can cause the volume of the haptic fluid lumen 106 to decrease. When the volume of the haptic fluid lumen 106 decreases, the fluid within the haptic fluid lumen 106 is moved or pushed into the optic fluid chamber 108. The optic portion 102 of the AIOL can change shape in response to fluid entering the optic fluid chamber 108 from the haptic fluid lumen 106. This can increase the base power or base spherical power of the AIOL and allow a patient with the AIOL implanted within the eye of the patient to focus on near objects. In this state, the adjustable AIOL can be considered to have undergone accommodation.

When the ciliary muscles relax, the peripheral region of the elastic capsular bag is stretched radially outward and the capsular bag elongates and more room is created within the capsular bag. The radially-outer haptic lumen wall 118 of the haptics 104 can be configured to respond to this capsular bag reshaping by returning to its non-deformed or non-stressed configuration. This causes the volume of the haptic fluid lumen 106 to increase or return to its non-deformed volume. This increase in the volume of the haptic fluid lumen 106 can cause the fluid within the optic fluid chamber 108 to be drawn out or otherwise flow out of the optic fluid chamber 108 and back into the haptic fluid lumen 106. Fluid moves out of the optic fluid chamber 108 into the haptic fluid lumen 106 through the same fluid channels 110 formed within the optic portion 102.

The optic portion 102 of the AIOL can change shape in response to fluid exiting the optic fluid chamber 108 and into the haptic fluid lumen 106. This can decrease the base power or base spherical power of the AIOL and allow a patient with the AIOL implanted within the eye of the patient to focus on distant objects or provide for distance vision. In this state, the AIOL can be considered to have undergone disaccommodation.

When the intraocular lens 100 is an AIOL, the radially-outer haptic lumen walls 118 of the haptics 104 can be made thinner than the radially-inner haptic lumen walls 120 to allow the haptics 104 to maintain a high degree of sensitivity to radial forces applied to an equatorial region of the haptics 104 by capsular bag reshaping as a result of ciliary muscle movements. The radially-inner haptic lumen walls 120 of the haptics 104 can be designed to be thicker or bulkier than the radially-outer haptic lumen walls 118 to provide the haptics 104 with stiffness or resiliency in the anterior-to-posterior direction. In certain embodiments, the radially-inner haptic lumen wall 120 can taper in shape as the radially-inner haptic lumen wall 120 gets closer to the optic portion 102. When designed in this manner, the haptics 104 can be less sensitive to capsular bag forces applied in the anterior-to-posterior direction. For example, when capsular bag forces are applied to the haptics 104 in the anterior-to-posterior direction, less fluid movement occurs between the haptic fluid lumens 106 and the optic fluid chamber 108 than when forces are applied in the radial direction. Since less fluid movement occurs, less changes in the base power of the AIOL occur.

Examples of AIOLs are discussed in the following U.S. patent publications: U.S. Pat. Pub. No. 2018/0153682 and in the following issued U.S. patents: U.S. Pat. Nos. 11,744,697; 11,660,182; 11,622,850; 11,426,270; 10,433,949; 10,299,913; 10,195,020; and 8,968,396, the contents of which are incorporated herein by reference in their entireties.

As will be discussed in more detail in relation to FIG. 1C, the intraocular lens 100 can also be a fluid-tunable non-accommodating IOL or a non-accommodating static-focus adjustable IOL. Examples of fluid-tunable non-accommodating IOLs or non-accommodating static-focus adjustable IOLs are discussed in U.S. Pat. No. 11,471,272, the content of which is incorporated herein by reference in its entirety.

In some embodiments, the intraocular lens 100 can be designed such that a gap 124 or void space radially separates the radially-inner haptic lumen wall 120 of the haptic 104 from the outer peripheral surface 122 of the optic portion 102.

In some embodiments, the fluid within the optic fluid chamber 108 and the haptic fluid lumen(s) 106 can be an oil. More specifically, in certain embodiments, the fluid within the optic fluid chamber 108 and the haptic fluid lumen(s) 106 can be a silicone oil or fluid. For example, the fluid can be a silicone polymer containing aliphatic or aromatic groups, or combinations thereof.

The fluid (e.g., the silicone oil) can be index-matched with a lens body material used to make the optic portion 102. When the fluid is index-matched with the lens body material, the entire optic portion 102 containing the fluid can act as a single lens. For example, the fluid can be selected so that it has a refractive index of between about 1.48 and 1.53 (or between about 1.50 and 1.53). In some embodiments, the fluid (e.g., the silicone oil) can have a polydispersity index of between about 1.2 and 1.3. In other embodiments, the fluid (e.g., the silicone oil) can have a polydispersity index of between about 1.3 and 1.5. In other embodiments, the fluid (e.g., the silicone oil) can have a polydispersity index of between about 1.1 and 1.2. Other example fluids are described in U.S. Patent Publication No. 2018/0153682, which is herein incorporated by reference in its entirety.

FIG. 1B illustrates an exploded view of the intraocular lens 100. The optic portion 102 of the intraocular lens 100 can comprise an anterior element 130 and a posterior element 132. A fluid-filled optic fluid chamber 108 can be defined in between the anterior element 130 and the posterior element 132.

The anterior element 130 can comprise an anterior outer surface 134 and an anterior inner surface opposite the anterior outer surface 134. The posterior element 132 can comprise a posterior outer surface and a posterior inner surface 140 opposite the posterior outer surface. Any of the anterior outer surface 134, the posterior optical surface, or a combination thereof can be considered and referred to as an external optical surface. The anterior inner surface and the posterior inner surface 140 can face the optic fluid chamber 108. At least part of the anterior inner surface and at least part of the posterior inner surface 140 can serve as chamber walls of the optic fluid chamber 108.

As shown in FIGS. 1B, the optic portion 102 can have an optical axis 142 extending in an anterior-to-posterior direction through a center of the optic portion 102. The optical axis can extend through the centers of both the anterior element 130 and the posterior element 132.

The thickness of the anterior element 130 can be greater at or near the optical axis 142 than at the periphery of the anterior element 130. In some embodiments, the thickness of the anterior element 130 can increase gradually from the periphery of the anterior element 130 toward the optical axis 142.

In certain embodiments, the thickness of the anterior element 130 at or near the optical axis 142 can be between about 0.45 mm and about 0.55 mm. In these and other embodiments, the thickness of the anterior element 130 near the periphery can be between about 0.20 mm and about 0.40 mm. Moreover, the anterior inner surface of the anterior element 130 can have less curvature or be flatter than the anterior outer surface 134.

The thickness of the posterior element 132 can be greater at or near the optical axis 142 than portions of the posterior element 132 radially outward from the optical axis 142 but prior to reaching a raised periphery 144 of the posterior element 132. The thickness of the posterior element 132 can gradually decrease from the optical axis 142 to portions radially outward from the optical axis 142 (but prior to reaching the raised periphery 144). As shown in FIG. 1B, the thickness of the posterior element 132 can increase once again from a radially inner portion of the raised periphery 144 to a radially outer portion of the raised periphery 144.

In certain embodiments, the thickness of the posterior element 132 at or near the optical axis 142 can be between about 0.45 mm and about 0.55 mm. In these and other embodiments, the thickness of the posterior element 132 radially outward from the optical axis 142 (but prior to reaching the raised periphery 144) can be between about 0.20 mm and about 0.40 mm. The thickness of the posterior element 132 near the radially outer portion of the raised periphery 144 can be between about 1.00 mm and 1.15 mm. Moreover, the posterior inner surface 140 of the posterior element 132 can have less curvature or be flatter than the posterior optical surface.

FIG. 1B also illustrates that each of the haptics 104 (e.g., any of the first haptic 104A or the second haptic 104B) can have a proximal attachment end 114 and a closed distal free end 116. A haptic fluid port 152 can be defined at the proximal attachment end 114 of the haptic 104. The haptic fluid port 152 can serve as a chamber opening of the haptic fluid lumen 106. Fluid within the haptic fluid lumen 106 can flow out of the haptic fluid lumen 106 through the haptic fluid port 152 and into the optic fluid chamber 108 via the pair of fluid channels 110 when the haptic 104 is coupled to the optic portion 102. Similarly, fluid within the optic fluid chamber 108 can flow out of the optic fluid chamber 108 through the pair of fluid channels 110 and into the haptic fluid lumen 106 through the haptic fluid port 152. A pair of outer apertures 156 and inner apertures 146 can serve as ends of the fluid channels 110.

As shown in FIG. 1B, each of the haptics 104 can be coupled to the optic portion 102 at the haptic-optic interface 112. More specifically, the proximal attachment end 114 can be coupled to the protruding outer surface 154 of the posterior element 132. The protruding outer surface 154 can also be referred to as a “landing” or “haptic attachment landing.” The protruding outer surface 154 can extend out radially from an outer peripheral surface 122 of the optic portion 102. For example, the protruding outer surface 154 can extend out radially from an outer peripheral surface 122 of the posterior element 132 of the optic portion 102. The protruding outer surface 154 can extend out radially from the outer peripheral surface 122 between about 10 microns and 1.0 mm or between about 10 microns and 500 microns.

The proximal attachment end 114 can have a substantially flat surface to adhere or otherwise couple to a substantially flat surface of the protruding outer surface 154. When the proximal attachment end 114 is coupled to the protruding outer surface 154, the haptic fluid port 152 can surround the outer apertures 156 of the fluid channels 110. The haptics 104 can be coupled or adhered to the optic portion 102 via biocompatible adhesives. In some embodiments, the adhesives can be the same adhesives used to couple or adhere the anterior element 130 to the posterior element 132.

FIG. 1C illustrates an exploded perspective view of another embodiment of an intraocular lens 100. The intraocular lens 100 shown in FIG. 1C can be a fluid-tunable non-accommodating intraocular lens.

The intraocular lens 100 can comprise an optic portion 102 and one or more haptics 104 extending from the optic portion 102. The haptics 104 can comprise a first haptic 104A and a second haptic 104B extending peripherally from or coupled to the optic portion 102. Each of the haptics 104 can comprise a kink 162 or bend defined along an arm of the haptic 104. The kink 162 or bend can allow the haptic 104 to compress or flex. Each of the haptics can terminate at a free or unconnected haptic distal end 116.

For example, the intraocular lens 100 can be a one-piece lens such that the haptics 104 are connected to and extend from the optic portion 102. In other embodiments, the haptics 104 are coupled to and adhered to the optic portion 102. For example, the haptics 104 can be adhered to the optic portion 102 after each is formed separately.

The optic portion 102 can comprise an anterior element 130, a posterior element 132, and an optic fluid chamber 108 defined in between the anterior element 130 and the posterior element 132. The optic fluid chamber 108 can be filled with a fluid.

In some embodiments, the fluid within the optic fluid chamber 108 can be an oil. More specifically, in certain embodiments, the fluid within the optic fluid chamber 108 can be a silicone oil.

The anterior element 130 can comprise an anterior outer surface 134. The anterior outer surface 134 can comprise a unique lens surface profile 164 or pattern defined on the anterior outer surface 134.

In some embodiments, the lens surface profile 164 can comprise a central diffractive area or structure comprising a plurality of diffractive zones or steps. In these and other embodiments, the widths of the diffractive zones can decrease in a radially outward manner such that zone widths at a periphery of the lens are smaller than zone widths near a central portion of the lens.

In certain embodiments, the lens surface profile 164 can split light into multiple foci or focal points. In these embodiments, the intraocular lens 100 can be considered a multifocal IOL or an adjustable multifocal IOL.

In some embodiments, the lens surface profile 164 can be configured to split light into two focal points (e.g., allowing for near and distant vision). In these embodiments, the intraocular lens 100 can be considered a bifocal IOL or an adjustable bifocal IOL.

The lens surface profile 164 can also be configured to split light into three focal points (e.g., allowing for near, intermediate, and distant vision). In these embodiments, the intraocular lens 100 can be considered a trifocal IOL or an adjustable trifocal IOL.

In other embodiments not shown in FIG. 1C, the external optical surface can have a uniformly curved (e.g., a spherical) lens surface or an aspherical lens surface providing focusing power for a single distance. In these embodiments, the intraocular lens 100 can be considered a monofocal IOL or an adjustable monofocal IOL.

In additional embodiments not shown in FIG. 1C, the anterior outer surface 134 can have a lens surface profile or pattern configured to provide an extended depth of focus or a single elongated focal point. In these embodiments, the intraocular lens 100 can be considered an extended depth of focus (EDOF) IOL or an adjustable EDOF IOL.

Moreover, any of the monofocal IOLs, the multifocal IOLs, or the EDOF IOLs can comprise a toric lens profile.

In some embodiments, the optic portion 102 of the intraocular lens 100 can have an optic portion diameter. The optic portion diameter can be between about 5.0 mm and 8.0 mm. For example, the optic portion diameter can be about 6.0 mm.

As will be discussed in more detail in the following sections, the intraocular lens components 101 of the intraocular lens 100 can be printed using certain 3D printing technologies that involve the curing of a photo-sensitive liquid intraocular lens formulation or a liquid resin by light energy.

The 3D printing technologies can comprise stereolithography (SLA), digital light processing (DLP), projection micro stereolithography (PμSL), and two photon polymerization (2PP).

One technical problem faced by the applicant is that most commercial 3D printing materials or resins do not possess suitable mechanical properties, clarity, or biocompatibility profiles for the manufacturing of IOLs. Similarly, existing IOL formulations are not suitable for 3D printing. One technical solution discovered and developed by the applicant is the intraocular lens formulation disclosed herein, which is not only suitable for 3D printing but also suitable for the printing of IOL components with intricate geometries.

In some embodiments, the intraocular lens formulation can comprise a plurality of monomers, a cross-linkable polymer comprising the plurality of monomers, a crosslinker, and a photoinitiator. The intraocular lens formulation can be in liquid form prior to being cured by light energy.

In some embodiments, the plurality of monomers can comprise an alkyl acrylate and/or alkyl methacrylate, a phenyl acrylate or phenyl methacrylate, and, optionally, a fluoromethacrylate or a fluoroacrylate.

In certain embodiments, the alkyl acrylate can be butyl acrylate (e.g., n-butyl acrylate), the alkyl methacrylate can be butyl methacrylate, the phenyl acrylate can be phenylethyl acrylate (e.g., 2-phenylethyl acrylate), the phenyl methacrylate can be phenylethyl methacrylate, the fluoromethacrylate can be trifluoroethyl methacrylate (e.g., 2,2,2-trifluoroethyl methacrylate), and the fluoroacrylate can be 2,2,2-trifluoroethyl acrylate. In alternative embodiments, the alkyl acrylate or alkyl methacrylate can be any of: octyl acrylate, nonyl acrylate, decyl acrylate, dodecyl methacrylate, n-hexyl acrylate, n-octyl methacrylate, n-butyl methacrylate, n-hexyl methacrylate, 2-ethylhexyl acrylate, 2-ethylhexyl methacrylate, 2,2-dimethylpropyl acrylate, 2,2-dimethylpropyl methacrylate, trimethylcyclohexyl acrylate, trimethylcyclohexyl methacrylate, isopropyl acrylate, isopropyl methacrylate, isobutyl acrylate, isobutyl methacrylate, isopentyl acrylate, isopentyl methacrylate, cyclohexyl acrylate, cyclohexyl methacrylate, cyclohexylmethyl acrylate, cyclohexylmethyl methacrylate, 2-cyclohexylethyl acrylate, 2-cyclohexylethyl methacrylate and mixtures thereof. In addition, alternatives for butyl acrylate may include a branched chain alkyl ester, e.g. 2-ethylhexyl acrylate, 2-ethylhexyl methacrylate, 2,2-dimethylpropyl acrylate, 2,2-dimethylpropyl methacrylate, trimethylcyclohexyl acrylate, trimethylcyclohexyl methacrylate, isobutyl acrylate, isobutyl methacrylate, isopentyl acrylate, isopentyl methacrylate, or mixtures thereof.

In additional embodiments, the phenyl acrylate or phenyl methacrylate can be any of: tribromophenyl acrylate, 2-(9H-Carazole-9-yl)ethyl methacrylate, 3-chlorostyrene, 4-chlorophenyl acrylate, benzyl acrylate, benzyl methacrylate, benzyl methacrylamide, n-vinylcarbazole, pentabromophenyl acrylate, and pentabromophenyl methacrylate, phenylethyl methacrylate, 3-phenylpropyl acrylate, 3-phenylpropyl methacrylate, or mixtures thereof.

In further embodiments, the fluoromethacrylate or fluoroacrylate can be any of: heptadecafluorodecyl acrylate, heptadecafluorodecyl methacrylate, hexafluorobutyl acrylate, hexafluorobutyl methacrylate, tetrafluoropropyl acrylate, tetrafluoropropyl methacrylate, octafluoropentyl acrylate, octafluoropentyl methacrylate, dodecafluoropheptyl acrylate, dodecafluoropheptyl methacrylate, heptafluorobutyl acrylate, heptafluorobutyl methacrylate trifluoroethyl acrylate, trifluoroethyl methacrylate, hexafluoro-iso-propyl acrylate, hexafluoro-iso-propyl methacrylate, pentafluorophenyl acrylate, pentafluorophenyl methacrylate, or mixtures thereof.

In some embodiments, the alkyl acrylate or the alkyl methacrylate can be between about 10% and 30% of the intraocular lens formulation (by weight percentage, wt %). As a more specific example, the alkyl acrylate or the alkyl methacrylate can be between about 9% and 19% of the intraocular lens formulation (by wt %).

In some embodiments, the phenyl acrylate or the phenyl methacrylate can be between about 30% and 60% of the intraocular lens formulation (by wt %). As a more specific example, the phenyl acrylate or the phenyl methacrylate can be between about 38% and 47% of the intraocular lens formulation (by wt %).

In embodiments where the intraocular lens formulation comprises the fluoromethacrylate or the fluoroacrylate, the fluoromethacrylate or the fluoroacrylate can be between about 0% and 20% of the intraocular lens formulation (by wt %). As a more specific example, the fluoromethacrylate or the fluoroacrylate can be between about 7% and 16% of the intraocular lens formulation (by wt %).

In some embodiments, the crosslinkable polymer can be between about 5% and 40% of the intraocular lens formulation (wt %). In certain embodiments, the crosslinkable polymer can be less than 40% of the intraocular lens formulation (wt %).

In some embodiments, the crosslinker can be ethylene glycol dimethacrylate (EGDMA). In certain embodiments, the crosslinker can be between about 0.1% and about 5.0% of the intraocular lens formulation (by wt %). As a more specific example, the crosslinker can be between about 0.20% and 0.90% of the intraocular lens formulation (by wt 17%).

In alternative embodiments, the crosslinker can be any of: diacrylates and dimethacrylates of ethylene glycol, diethylene glycol, triethylene glycol, tetracthylene glycol, polyethylene glycol, butylene glycol, neopentyl glycol, hexane-1,6-diol and thio-diethylene glycol, or trimethylolpropane triacrylate, N,N′-dihydroxyethylene bisacrylamide, diallyl phthalate, triallyl cyanurate, divinylbenzene; ethylene glycol divinyl ether, N,N′-methylene-bis-(meth) acrylamide, sulfonated divinylbenzene, divinylsulfone, ethylene glycol diacrylate, 1,6 hexanediol diacrylate, dicyclopentyldimethylene diacrylate, trifunctional acrylates, trifunctional methacrylates, tetrafunctional acrylates, tetrafunctional methacrylates, or mixtures thereof

In some embodiments, the photoinitiator can be phenylbis(2,4,6-trimethylbenzoyl)-phosphincoxide, also known as Irgacure® 819. In certain embodiments, the photoinitiator can be between about 0.1% and about 5.0% of the intraocular lens formulation (by wt %). As a more specific example, the photoinitiator can be between about 2.5% and 4.5% of the intraocular lens formulation (by wt %).

The amount of the photoinitiator can depend on the total of all other ingredients in the intraocular lens formulation. In these embodiments, the photoinitiator can be less than about 5.0% of the intraocular lens formulation (by wt %).

In alternative embodiments, other photoinitiators can also be used such as camphorquinone with 1-phenyl-1,2-propanedione and 2-ethylhexyl-4-(dimethylamino)benzoate.

In some embodiments, the crosslinkable polymer can comprise the alkyl acrylate and/or the alkyl methacrylate, the phenyl acrylate or the phenyl methacrylate, a monomer comprising a hydroxyl moiety, a curing agent, and, optionally, the fluoromethacrylate or the fluoroacrylate.

In certain embodiments, the alkyl acrylate can be butyl acrylate (e.g., n-butyl acrylate), the alkyl methacrylate can be butyl methacrylate, the phenyl acrylate can be phenylethyl acrylate (e.g., 2-phenylethyl acrylate), the phenyl methacrylate can be phenylethyl methacrylate, the fluoromethacrylate can be trifluoroethyl methacrylate (e.g., 2,2,2-trifluoroethyl methacrylate), and the fluoroacrylate can be trifluoroethyl acrylate.

In some embodiments, the alkyl acrylate or the alkyl methacrylate can be between about 40% and about 45% of the crosslinkable polymer (by wt %). As a more specific example, the alkyl acrylate or the alkyl methacrylate can be between about 42% and about 44% of the crosslinkable polymer (by wt %).

In some embodiments, the phenyl acrylate or the phenyl methacrylate can be between about 25% and about 35% of the crosslinkable polymer (by wt %). As a more specific example, the phenyl acrylate or the phenyl methacrylate can be between about 28% and about 32% of the crosslinkable polymer (by wt %).

In some embodiments, the fluoromethacrylate or the fluoroacrylate can be between 20% and 25% of the crosslinkable polymer (by wt %). As a more specific example, the fluoromethacrylate or the fluoroacrylate can be between 21% and 23% of the crosslinkable polymer (by wt %).

In some embodiments, the monomer comprising the hydroxyl moiety in the crosslinkable polymer can be 2-hydroxyethyl acrylate (HEA).

In certain embodiments, the monomer comprising the hydroxyl moiety can be between about 0.5% and about 2.0% of the crosslinkable polymer (by wt %). As a more specific example, the monomer comprising the hydroxyl moiety can be between about 1.0% and about 1.5% of the crosslinkable polymer (by wt %).

In some embodiments, the curing agent in the crosslinkable polymer can be a photoinitiator. For example, the curing agent for the crosslinkable polymer can be a mixture of diphenyl(2,4,6-trimethylbenzoyl) phosphine oxide with 2-hydroxy-2-methylpropiophenone, commonly known as Darocur® 4265.

In other embodiments, the curing agent can be a thermal initiator, for example di(4-tert-butylcyclohexyl) peroxydicarbonate (Perkadox® 16).

In certain embodiments, the curing agent can be between about 1.0% and about 3.0% of the crosslinkable polymer (by wt %). As a more specific example, the curing agent can be between 1.50% and 2.0% of the crosslinkable polymer (by wt %).

In some embodiments, the plurality of monomers and the crosslinker of the intraocular lens formulation can be passed through a column of basic alumina to remove polymerization inhibitors prior to being added to the intraocular lens formulation. The basic alumina column can have a pH of approximately 9.7±0.3. The column can be a disposable single-use column. The basic alumina column can comprise particles having a particle size between about 40 to 65 μm and a pore size of between 60-80 Angstroms.

In some embodiments, each of the plurality of monomers and the crosslinker can be passed through the basic alumina column without a solvent.

In some embodiments, each of the alkyl acrylate or the alkyl methacrylate (e.g., n-butyl acrylate), the phenyl acrylate or the phenyl methacrylate (e.g., 2-phenylethyl acrylate), the fluoromethacrylate or the fluoroacrylate (e.g., 2,2,2-trifluoroethyl methacrylate), and the crosslinker (e.g., the ethylene glycol dimethacrylate (EGDMA)) can be passed through the column of the basic alumina to remove polymerization inhibitors prior to being added as part of the intraocular lens formulation.

One unexpected discovery made by the applicant is that removing polymerization inhibitors bypassing the monomers and the crosslinker through the column of basic alumina made the intraocular lens formulation more suitable for 3D printing. More specifically, the applicant discovered that passing the monomers and the crosslinker through the column of basic alumina removed certain inhibitors (such as monomethyl ether hydroquinone or MEHQ) from the constituents that made the overall intraocular lens formulation more suitable for 3D printing. Inhibitor removal may also be accomplished by vacuum distillation or washing the monomers with dilute aqueous base solution.

In some embodiments, the printed intraocular lens component can have a refractive index between about 1.48 and about 1.53. In certain embodiments, the refractive index of the printed intraocular lens component can be between about 1.50 and about 1.53.

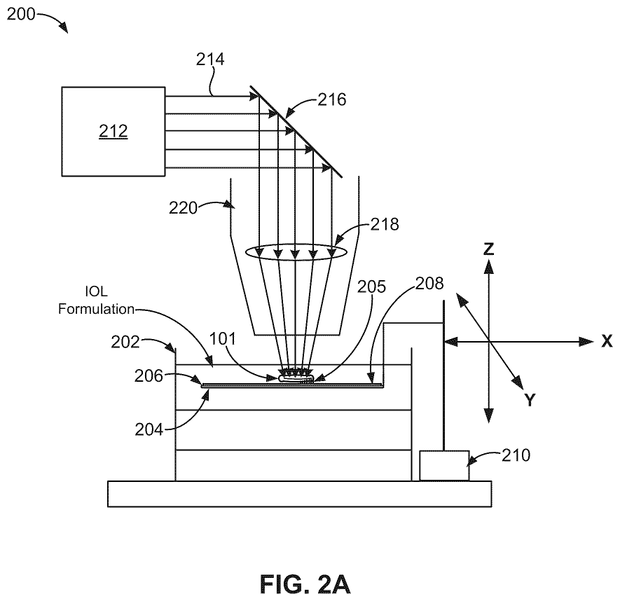

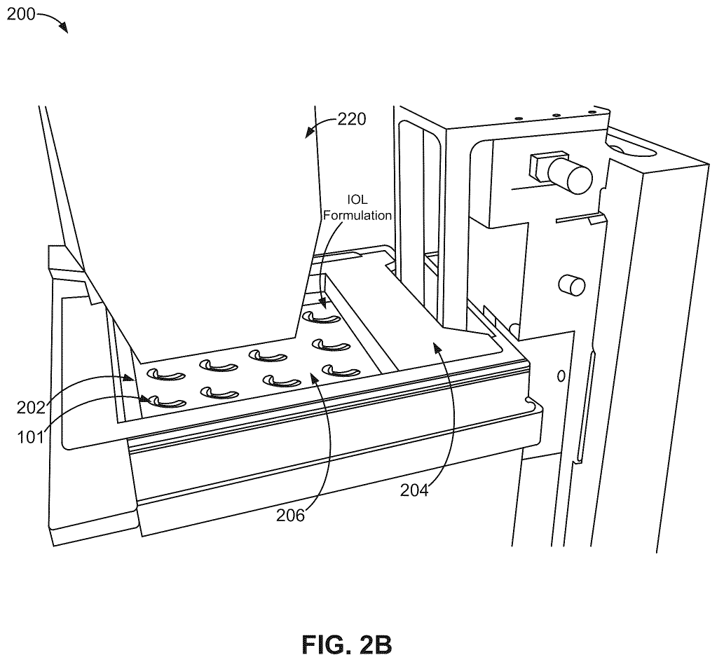

FIG. 2A is a schematic diagram illustrating a 3D printer 200 for printing an intraocular lens component 101. In some embodiments, the intraocular lens component 101 can be a haptic 104 (see, e.g., FIGS. 1A-1C) of an intraocular lens 100.

The 3D printer 200 can comprise a reservoir 202 or resin reservoir configured to receive and contain the intraocular lens formulation. The intraocular lens formulation can be in liquid form when introduced (e.g., poured, injected, pumped, etc.) into the reservoir 202.

The reservoir 202 can be an open or semi-open container or tank for receiving and containing the intraocular lens formulation. In some embodiments, the reservoir 202 can also be sized to accommodate at least part of the intraocular lens component 101 while the intraocular lens component 101 is being 3D printed. The reservoir 202 can also be equipped to keep the intraocular lens formulation under an inert atmosphere, for example, under nitrogen or argon gas.

The 3D printer 200 can also comprise a build platform 204 comprising a build surface 206 or build plate surface. The build surface 206 can be configured to be initially immersed in or otherwise in fluid contact with the intraocular lens formulation within the reservoir 202.

In some embodiments, the 3D printer 200 can further comprise a borosilicate or quartz glass plate 208 coupled to the build surface 206 and the intraocular lens component 101 can be printed directly on the glass plate 208. For example, the glass plate 208 can be a borosilicate plate adhered or otherwise affixed to the build surface 206. The glass plate 208 can allow for improved adhesion during printing and can allow the printed intraocular lens component 101 to be easily released after the printing process is complete.

In some embodiments, the build surface 206 can be made of a polymeric material such as polypropylene, polyether ether ketone, polyoxymethylene or polyetherimide.

During the 3D printing build process, the reservoir 202, the build platform 204, or a combination thereof can be translated in a z-direction after each layer of the intraocular lens component 101 is printed. For example, the build platform 204, the reservoir 202, or a combination thereof can be translatable in a z-direction via one or more linear actuators 210 (e.g., stepper motors and drivers). As a more specific example, the build platform 204, the reservoir 202, or a combination thereof can be translated vertically downward (i.e., in a z-direction) after each layer of the intraocular lens component 101 is printed.

In some embodiments, the build platform 204 and the build surface 206 can be configured to translate or translatable in the x,y plane. In these embodiments, the build platform 204 can be translatable in an x-direction and/or a y-direction via one or more mechanical actuators or drivers.

The 3D printer 200 can also comprise a light source 212 or light projector configured to generate light 214 or light energy. In some embodiments, the light generated by the light source 212 can be ultraviolet (UV) light. As a more specific example, the light source 212 can comprise a number of UV light-emitting diodes (LEDs).

The 3D printer 200 can further comprise one or more mirrors 216 and one or more projection optics 218 or imaging optics configured to direct the light generated by the light source 212 at the intraocular lens formulation within the reservoir 202 to cure a portion of the intraocular lens formulation and form a layer of the intraocular lens component 101 on the build surface 206 or the glass plate 208.

The one or more projection optics 218 can be positioned in between the light source 212/mirror(s) 216 and the reservoir 202 to focus the light 214 and increase the print resolution. Although FIG. 2A shows the light source 212, the one or more mirrors 216, and the one or more projection optics 218 as separate units, it is contemplated by this disclosure and it should be understood by one of ordinary skill in the art that the light source 212, the one or more mirrors 216, and the one or more projection optics 218, or some combination thereof, can be integrated into one projection unit 220 (see, e.g., FIG. 2B).

In certain embodiments, the UV light generated by the light source 212 can have a wavelength of between 365 nm and 410 nm. More specifically, the UV light generated by the light source 212 can have a wavelength of about 365 nm, 385 nm, or 405 nm.

As shown in FIG. 2A, the light 214 generated by the light source 212 can be directed at the intraocular lens formulation within the reservoir 202 in a top-down manner. This can minimize the number of support structures 205 needed to maintain the stability of the intraocular lens component 101 as the intraocular lens component 101 is being printed.

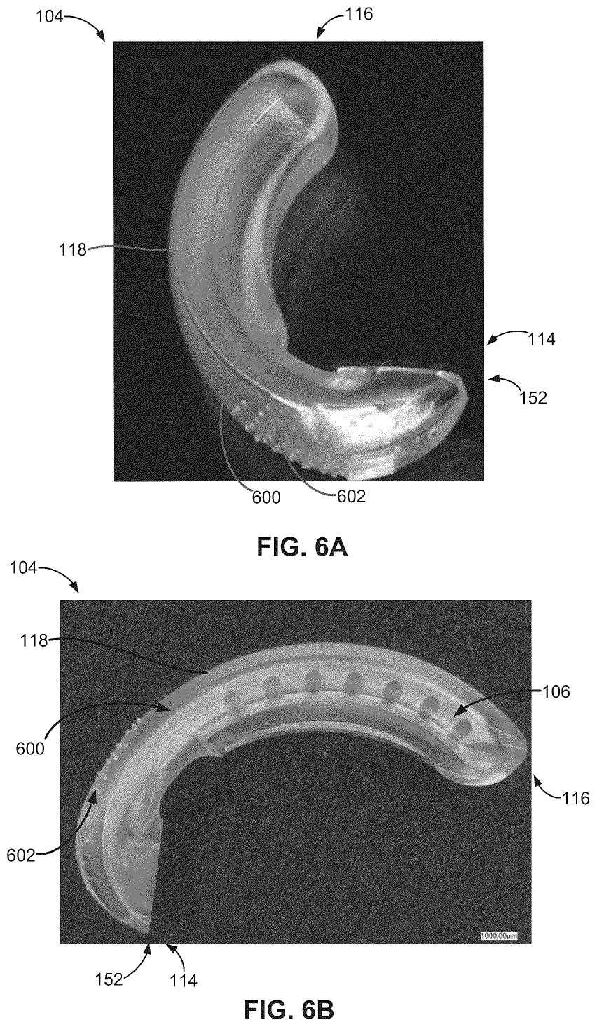

As shown in FIG. 2A, a plurality of support structures 205 may be needed to maintain the stability of the intraocular lens component 101 as the intraocular lens component 101 is being 3D printed. For example, the support structures 205 can be in the form of thin strands, ribs, or lattice-like structures that extend from an exterior surface of the intraocular lens component 101 at one end and attach to the build surface 206 (or the glass plate 208) at the other end. The support structures 205 can be made of the same material as the 3D-printed intraocular lens component 101 (i.e., the support structures 205 can also be made from a cured instance of the intraocular lens formulation). As will be discussed in more detail in relation to FIGS. 6A and 6B, the support structures 205 can be post-processed (e.g., cut, clipped, or trimmed) in a way to produce support structure remnants that help with enhancing the rotational stability of the intraocular lens component 101 when the component is implanted within the eye of a subject.

In some embodiments, the 3D printer 200 can also comprise a digital micromirror device (DMD) comprising a plurality of micromirrors arranged in a matrix that can be manipulated to generate an image pattern that can be used to print the intraocular lens component 101.

In some embodiments, the 3D printer 200 can be controlled by a digital controller and/or a computing device communicatively coupled to the digital controller.

In some embodiments, the 3D printer 200 can be referred to as a projection micro stereolithography (PμSL) printer.

FIG. 2B illustrates one embodiment of a PμSL 3D printer 200 for printing the intraocular lens component 101. The 3D printer 200 can comprise a reservoir 202 or resin reservoir configured to receive and contain the photosensitive intraocular lens formulation.

The 3D printer 200 can comprise a build platform 204 having a build surface 206 or build plate surface. The build surface 206 can be immersed in or otherwise in fluid contact with the photosensitive intraocular lens formulation within the reservoir 202.

In some embodiments, the 3D printer 200 can comprise a glass plate or surface coupled to the build surface 206 and the intraocular lens component 101 can be formed directly on the glass plate or surface.

The reservoir 202, the build platform 204, or a combination thereof can be configured to be translated in a z-direction (e.g., vertically downward) after each layer of the intraocular lens component 101 is printed. In certain embodiments, the reservoir 202, the build platform 204, or a combination thereof can be configured to be translated in an x-direction and/or a y-direction after each layer of the intraocular lens component 101 is printed.

The PμSL 3D printer 200 can further comprise a projection unit 220 or projection light unit configured to direct UV light at the intraocular lens formulation within the reservoir 202 from a top-down position. In some embodiments, the projection unit 220 can comprise at least part of a light source 212 (e.g., UV LEDs) or light projector configured to generate the UV light and one or more mirrors 216 and projection optics 218 configured to direct the light generated by the light source 212 at the intraocular lens formulation within the reservoir 202 to cure a portion of the intraocular lens formulation and form a layer of the intraocular lens component 101 on the build surface 206 or the glass plate. The UV light generated by the light source 212 can have a wavelength of about 405 nm.

The PμSL 3D printer 200 can also comprise a digital micromirror device (DMD). The 3D printer 200 can be controlled by a digital controller and/or a computing device communicatively coupled to the digital controller.

FIG. 2B also illustrates that multiple intraocular lens components 101 can be printed simultaneously on the build surface 206 or the glass plate. For example, the PμSL 3D printer 200 can be used to print multiple IOL haptics 104 (see, e.g., FIG. 1A-1C) simultaneously on the build surface 206 or the glass plate.

Although not shown in FIG. 2B, the PμSL 3D printer 200 can also comprise a hood or cover configured to cover or contain the reservoir 202, the build platform 204, and/or the projection unit 220.

The PμSL 3D printer 200 can print the intraocular lens component 101 using the print parameters listed in Table 1 below:

TABLE 1
Print Parameters

| | |
|---|---|
| Light Intensity: | 1 mW/cm² to 500 mW/cm² |
| Exposure Time (per exposure): | 0.1 seconds to 10 seconds |
| Viscosity: | 1 cPs to 1200 cPs |
| Dwell/Wait times (idle times) | 1 second to 900 seconds |
| before and after exposure: | |
| Layer Thickness: | 5 μm to 50 μm |
| Heater Temperature: | 15° C. to 50° C. |
| Vat/Printhead Velocity: | 0.1 mm/sec to 25 mm/sec |
| Acceleration: | 1 mm/sec² to 300 mm/sec² |

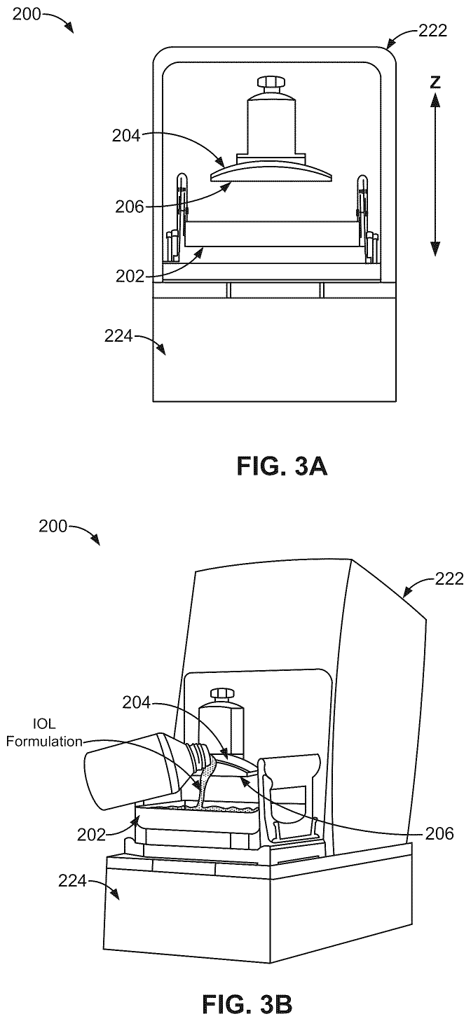

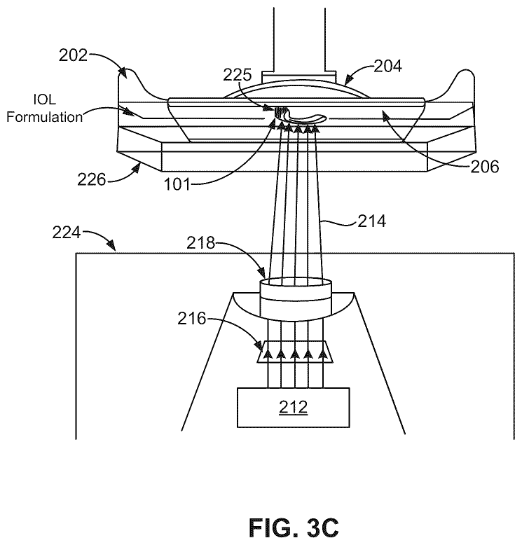

FIGS. 3A and 3B illustrate another embodiment of a 3D printer 200 for printing the intraocular lens component 101. The 3D printer 200 can comprise a reservoir 202 or resin reservoir configured to receive and contain the intraocular lens formulation.

As shown in FIG. 3B, the intraocular lens formulation can be in liquid form when poured or otherwise introduced (e.g., injected, pumped, etc.) into the reservoir 202. As will be discussed in more detail in the following sections, at least part of the base or bottom surface of the reservoir 202 can be clear or transparent such that light can penetrate through the base or bottom surface of the reservoir 202. This can allow light generated by a light source 212 (see, e.g., FIG. 3C) below the reservoir 202 to reach the intraocular lens formulation within the reservoir 202.

The 3D printer 200 can also comprise a build platform 204 comprising a build surface 206 or build plate surface positioned above the reservoir 202. The build surface 206 of the build platform 204 can be lowered into the reservoir 202 such that at least part of the build surface 206 is immersed or otherwise in fluid contact with the intraocular lens formulation within the reservoir 202 when the printing process begins.

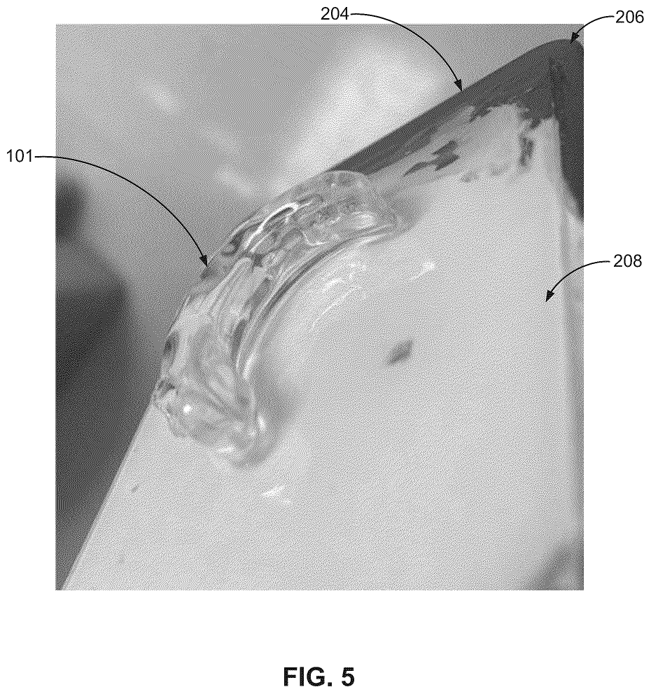

In some embodiments, the 3D printer 200 can further comprise a glass plate 208 (see, e.g., FIG. 5) coupled to the build surface 206 and the intraocular lens component 101 can be printed directly on the glass plate 208. For example, the glass plate 208 can be a borosilicate plate adhered or otherwise affixed to the build surface 206.

In some embodiments, the build surface 206 can be made of a polymeric material such as polypropylene, polyether ether ketone, polyoxymethylene or polyetherimide.

During the 3D printing process, the build platform 204 can be translated in a z-direction after each layer of the intraocular lens component 101 is printed. For example, the build platform 204 can be translatable in a z-direction via one or more linear actuators (e.g., stepper motors and drivers). As a more specific example, the build platform 204 can be translated vertically upward (i.e., in a z-direction) after each layer of the intraocular lens component 101 is printed.

In some embodiments, the build platform 204 can also be configured to translate in the x,y plane. In these embodiments, the build platform 204 can be translatable in an x-direction and/or a y-direction via one or more mechanical actuators or drivers.

The 3D printer 200 can further comprise a printer hood 222 or cover and a printer base 224. The printer hood 222 or cover can be configured to cover or contain the reservoir 202 and the build platform 204 during the printing process.

The printer base 224 can be a housing or support platform positioned vertically below the reservoir 202. The printer base 224 can house or contain at least part of the light source 212, one or more mirrors 216, and one or more projection optics 218 or imaging optics (see, e.g., FIG. 3C).

FIG. 3C is a schematic diagram illustrating part of the 3D printer 200 of FIG. 3A in operation. FIG. 3C illustrates a cross-section of the reservoir 202 containing the intraocular lens formulation and part of the build platform 204 comprising the build surface 206 immersed in the intraocular lens formulation within the reservoir 202. Also shown is a schematic representation of the light source 212 within the printer base 224 generating light 214 or light energy directed at the intraocular lens formulation within the reservoir 202 in order to cure a layer of the intraocular lens component 101 on the build surface 206.

The light source 212 or light projector can be configured to generate light 214 or light energy used to cure or photopolymerize the intraocular lens formulation. In some embodiments, the light generated by the light source 212 can be ultraviolet (UV) light. As a more specific example, the light source 212 can comprise a number of UV light-emitting diodes (LEDs).

As shown in FIG. 3C, at least part of a reservoir base 226 or bottom surface of the reservoir 202 can be clear/transparent or UV transmissible such that UV light generated by the light source 212 can penetrate through the reservoir base 226 or bottom surface of the reservoir to allow the UV light to reach the intraocular lens formulation within the reservoir 202. The printer base 224 can further comprise one more mirrors 216 and one or more projection optics 218 or imaging optics configured to direct the light generated by the light source 212 at the intraocular lens formulation within the reservoir 202 to cure a portion of the intraocular lens formulation and form a layer of the intraocular lens component 101 on the build surface 206 or the glass plate 208.

The one or more projection optics 218 can be positioned in between the light source 212/mirror(s) 216 and the reservoir 202 to focus the light 214 and increase the print resolution.

In certain embodiments, the UV light generated by the light source 212 can have a wavelength of between 365 nm and 410 nm. More specifically, the UV light generated by the light source 212 can have a wavelength of about 405 nm, 385 nm, or 365 nm.

As shown in FIG. 3C, the light 214 generated by the light source 212 can be directed at the intraocular lens formulation within the reservoir 202 in a bottom-up manner. A plurality of support structures 205 may be needed to maintain the stability of the intraocular lens component 101 as the intraocular lens component 101 is being printed. For example, the support structures 205 can be in the form of thin strands, ribs, or lattice-like structures that extend from an exterior surface of the intraocular lens component 101 at one end and attach to the build surface 206 (or the glass plate 208) at the other end. The support structures 205 can be made of the same material as the 3D-printed intraocular lens component 101 (i.e., the support structures 205 can also be made from a cured instance of the intraocular lens 19 formulation). As will be discussed in more detail in relation to FIGS. 6A and 6B, the support structures 205 can be post-processed (e.g., cut, clipped, or trimmed) in a way to produce support structure remnants that help with enhancing the rotational stability of the intraocular lens component 101 when the component is implanted within the eye of a subject.

As shown in FIG. 3C, the build surface 206 can be configured to be immersed in or otherwise in fluid contact with the intraocular lens formulation within the reservoir 202 during the printing process.

In some embodiments, the 3D printer 200 can also comprise a digital micromirror device (DMD) comprising a plurality of micromirrors arranged in a matrix that can be manipulated to generate an image pattern that can be used to print the intraocular lens component 101.

In some embodiments, the 3D printer 200 can be controlled by a digital controller and/or a computing device communicatively coupled to the digital controller.

In some embodiments, the 3D printer 200 shown in FIGS. 3A, 3B, and 3C can be referred to as a digital light processing (DLP) 3D printer.

Although FIGS. 3A-3C and FIGS. 2A-2B illustrate specific types of 3D printers (e.g., DLP 3D printers and PμSL 3D printers, respectively), it is contemplated by this disclosure and it should be understood by one of ordinary skill in the art that other types of 3D printers can also be used to print the intraocular lens component 101 using the intraocular lens formulation disclosed herein as long as the 3D printer is able to print at a resolution of 30 μm or better.

For example, different types of stereolithography (SLA) 3D printers and a two photon polymerization (2PP) can also be used to 3D print the intraocular lens component 101 using the intraocular lens formulation disclosed herein.

In some embodiments, the intraocular lens component 101 can be printed using the intraocular lens formulation and the 3D printer disclosed in U.S. Pat. No. 11,298,874, the content of which is incorporated herein by reference in its entirety.

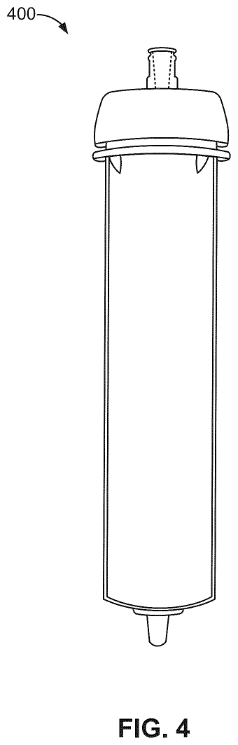

FIG. 4 illustrates one embodiment of a basic alumina column 400 that can be used to filter out inhibitors from monomers of the intraocular lens formulation.

In some embodiments, the monomers of the intraocular lens formulation including the alkyl acrylate or the alkyl methacrylate (e.g., n-butyl acrylate), the phenyl acrylate or the phenyl methacrylate (e.g., 2-phenylethyl acrylate), and, optionally, the fluoromethacrylate or the fluoroacrylate (e.g., 2,2,2-trifluoroethyl methacrylate) can be passed through the basic alumina column 400 to remove certain inhibitors (such as monomethyl ether hydroquinone or MEHQ) from the monomers prior to use.

In some embodiments, the crosslinker (e.g., the ethylene glycol dimethacrylate (EGDMA)) can also be passed through the basic alumina column 400 to remove any inhibitors prior to use.

The plurality of monomers and the crosslinker of the intraocular lens formulation can be passed through the basic alumina column 400 prior to being added to or incorporated into the intraocular lens formulation.

In some embodiments, the basic alumina column 400 can have a pH of approximately 9.7±0.3. The basic alumina column 400 can be a disposable or single-use column.

The basic alumina column 400 can comprise particles having a particle size between about 40 to 65 μm and a pore size of between 60-80 Angstroms.

In some embodiments, each of the plurality of monomers and the crosslinker can be passed through the basic alumina column 400 without a solvent.

FIG. 5 illustrates one embodiment of an intraocular lens component 101 printed on a glass plate 208 coupled to a build surface 206. As shown in FIG. 5, the intraocular lens component 101 can be a haptic 104 of an intraocular lens 100 (see, e.g., FIGS. 1A and 1B). The intraocular lens component 101 can be printed directly on the glass plate 208 coupled or adhered to the build surface 206 of the build platform 204.

The intraocular lens component 101 can be printed based on a computer-aided design (CAD) model of the intraocular lens component 101 stored as part of a CAD file. The CAD file can be sliced into a series of two-dimensional (2D) images that depict cross-sectional layers of the intraocular lens component 101. The 2D images can also be referred to as digital masks. Each layer of the intraocular lens component 101 can be printed on top of an immediately preceding layer.

The intraocular lens component 101, once printed, can be rinsed with isopropyl alcohol (IPA) or isopropanol to remove unreacted monomers. In some embodiments, the printed intraocular lens component 101 can be rinsed with 99% (vol. %) IPA (or 95%, 96%, 97% or 98% IPA).

The printed intraocular lens component 101 can also be further processed in one or more post-processing steps such as additional curing, polishing, and deburring.

FIG. 6A illustrates a top perspective view of a haptic 104 of an intraocular lens 3D printed using one of the 3D printers 200 (see, e.g., FIGS. 2A-2B or FIGS. 3A-3C) disclosed herein. For example, the haptic 104 shown in FIG. 6A can be printed using a projection micro-stereolithography (PμSL) 3D printer.

The 3D-printed haptic 104 can comprise a haptic body having a radially-outer haptic surface 600. The radially-outer haptic surface 600 can be a radially-outer surface of the radially-outer haptic lumen wall 118 of the haptic 104.

The 3D-printed haptic 104 can also comprise a plurality of 3D-printing support structure remnants 602 protruding or otherwise extending laterally outward from the radially-outer haptic surface 600. The 3D-printing support structure remnants 602 can be formed or made by removing portions of certain 3D-printing support structures 205 (see, e.g., FIG. 2A or FIG. 3C) used to support at least part of the 3D-printed haptic 104 during the 3D printing process. For example, the 3D-printing support structure remnants 602 can be formed or made by cutting, clipping, or trimming portions of the 3D-printing support structures 205 (see, e.g., FIG. 2A or FIG. 3C) used to support at least part of the 3D-printed haptic 104 during the 3D printing process.

FIG. 6B illustrates a top plan view of the 3D-printed haptic 104 shown in FIG. 6A. The haptic 104 can have a distal free end 116 and a proximal attachment end 114 opposite the distal free end 116. As shown in FIGS. 6A and 6B, the plurality of 3D-printing support structure remnants 602 can protrude from an area of the radially-outer haptic surface 600 proximal to the proximal attachment end 114. For example, the area of the radially-outer haptic surface 600 proximal to the proximal attachment end 114 can be located closer to the proximal attachment end 114 than the distal free end 116. For example, the plurality of 3D-printing support structure remnants 602 can be clustered or scattered along a segment of the haptic 104 (e.g., scattered along part of the radially-outer haptic surface 600) near or approaching the proximal attachment end 114.

The 3D-printing support structure remnants 602 can be shaped substantially as discrete bumps or nubs protruding from the radially-outer haptic surface 600. The 3D-printing support structure remnants 602 can be made of the same material as the haptic body of the 3D-printed haptic 104 (i.e., the 3D-printing support structure remnants 602 can be made of a cured instance of the intraocular lens formulation).

In some embodiments, a minimum height of each of the 3D-printing support structure remnants 602 can be about 10 μm. A maximum height of each of the 3D-printing support structure remnants 602 can be about 1000 μm. In other embodiments, the maximum height of each of the 3D-printing support structure remnants 602 can be greater than 1000 μm.

The 3D-printed haptic 104 can also comprise a haptic fluid lumen 106 extending through at least part of a haptic body of the 3D-printed haptic 104. The haptic fluid lumen 106 can be surrounded by the radially-outer haptic lumen wall 118 and a radially-inner haptic lumen wall. The radially-outer haptic surface 600 can be a radially-outer surface of the radially-outer haptic lumen wall 118.

The 3D-printed haptic 104 can also have a haptic fluid port 152 defined at the proximal attachment end 114. The haptic fluid port 152 can be in fluid communication with the haptic fluid lumen 106. The 3D-printing support structure remnants 602 can be located along an area of the radially-outer haptic surface 600 approaching or near the proximal attachment end 114.

In some embodiments, the 3D-printed haptic 104 can be used as part of an accommodating intraocular lens (AIOL). One of the advantages of fabricating the haptic 104 using 3D printing is that the 3D printing process can allow a manufacturer to produce an AIOL haptic with extremely intricate internal geometries in a single step without the need for separate mold tooling steps, machining operations, and core dissolution steps. The haptic 104 shown in FIGS. 6A and 6B can be printed using the intraocular lens formulation disclosed herein.

A method of 3D printing a haptic 104 of an intraocular lens can comprise 3D printing the haptic 104 of the intraocular lens. At least part of the haptic 104 can be supported by 3D-printing support structures (e.g., support structures 205, see FIG. 2A or 3C) during the 3D printing process. The method can also comprise removing portions of the 3D-printing support structures until a plurality of 3D-printing support structure remnants 602 remain along a surface (e.g., the radially-outer haptic surface 600) of the haptic 104.

In some embodiments, removing the portions of the 3D-printing support structures 205 can further comprise cutting, clipping, or trimming the 3D-printing support structures until only the 3D-printing support structure remnants 602 remain along the surface of the haptic 104.

In certain embodiments, 3D printing the haptic 104 of the intraocular lens can further comprise 3D printing the haptic 104 using a digital light processing (DLP) 3D printer.

In other embodiments, 3D printing the haptic 104 of the intraocular lens can further comprise 3D printing the haptic 104 using a projection micro-stereolithography (PμSL) 3D printer.

One technical problem faced by the applicant is how to design a haptic that improves the rotational stability of an intraocular lens comprising the haptic when the intraocular lens is implanted within an eye of the subject/patient. One technical solution discovered and developed by the applicant is the 3D-printed haptic disclosed herein comprising a plurality of 3D-printing support structure remnants protruding from a radially-outer haptic surface of the haptic located near a proximal attachment end of the haptic. The 3D-printing support structure remnants can be formed by cutting, clipping, or trimming portions of certain 3D-printing support structures used to support a part of the 3D-printed haptic during the 3D printing process.

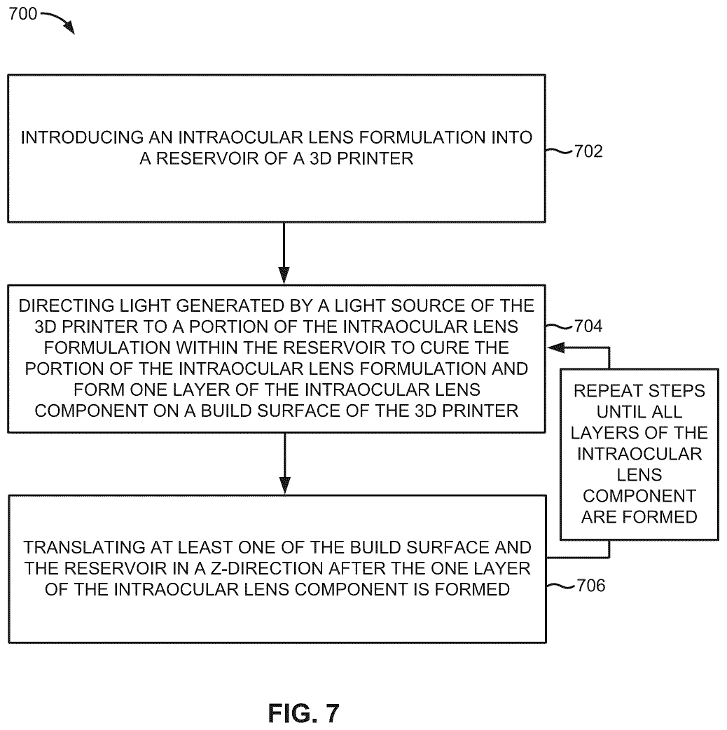

FIG. 7 is a flowchart illustrating one embodiment of a method 700 of 3D printing an intraocular lens component 101. The method 700 can comprise introducing an intraocular lens formulation into a reservoir 202 of a 3D printer 200 in step 702. The intraocular lens formulation can be in liquid form when introduced into the reservoir 202.

In some embodiments, the intraocular lens formulation can comprise a plurality of monomers, a crosslinker, a crosslinkable polymer comprising the plurality of monomers, and a photoinitiator. The plurality of monomers can comprise an alkyl acrylate and/or alkyl methacrylate, a phenyl acrylate or phenyl methacrylate, and, optionally, a fluoromethacrylate or a fluoroacrylate.

The method 700 can also comprise directing light 214 generated by a light source 212 of the 3D printer 200 to a portion of the intraocular lens formulation within the reservoir 202 to cure the portion of the intraocular lens formulation and form one layer of the intraocular lens component 101 on a build surface 206 of the 3D printer 200 in step 704.

In some embodiments, the light 214 generated by the light source 212 can be UV light. For example, the wavelength of the UV light can be between about 365 nm and about 410 nm (e.g., 405 nm).

In some embodiments, the exposure time of the intraocular lens formulation to the light 214 can be between about 0.1 seconds and about 10.0 seconds. The method 700 can also comprise waiting between 1 second and 900 seconds in between light exposures.

In certain embodiments, step 704 can also comprise adhering or otherwise coupling a glass plate 208 to the build surface 206 and forming the layer of the intraocular lens component 101 directly on the glass plate 208.

The method 700 can further comprise translating at least one of the build surface 206 and the reservoir 202 in a z-direction after the one layer of the intraocular lens component 101 is formed in step 706.

In some embodiments, each layer of the intraocular lens component 101 can have a thickness of between 5 μm and 50 μm. In certain embodiments, each layer of the intraocular lens component 101 can be printed in about 10 seconds. In other embodiments, each layer of the intraocular lens component 101 can be printed in between 10 seconds and 20 minutes.

The method 700 can further comprise repeating steps 704 and 706 until all layers of the intraocular lens component 101 are formed. In some embodiments, the intraocular lens component 101 can be a haptic of an intraocular lens. When the intraocular lens component 101 is a haptic, the total print time can be between about 10 minutes and 300 minutes.

In some embodiments, the 3D printer 200 can be a photopolymerizing 3D printer capable of printing at a print resolution of between about 2 μm and 30 μm. For example, the 3D printer can be a digital light processing (DLP) 3D printer or a projection micro-stereolithography (PμSL) 3D printer.

In other embodiments, the 3D printer can be another type of stereolithography 3D printer or a two photon polymerization (2PP) 3D printer.

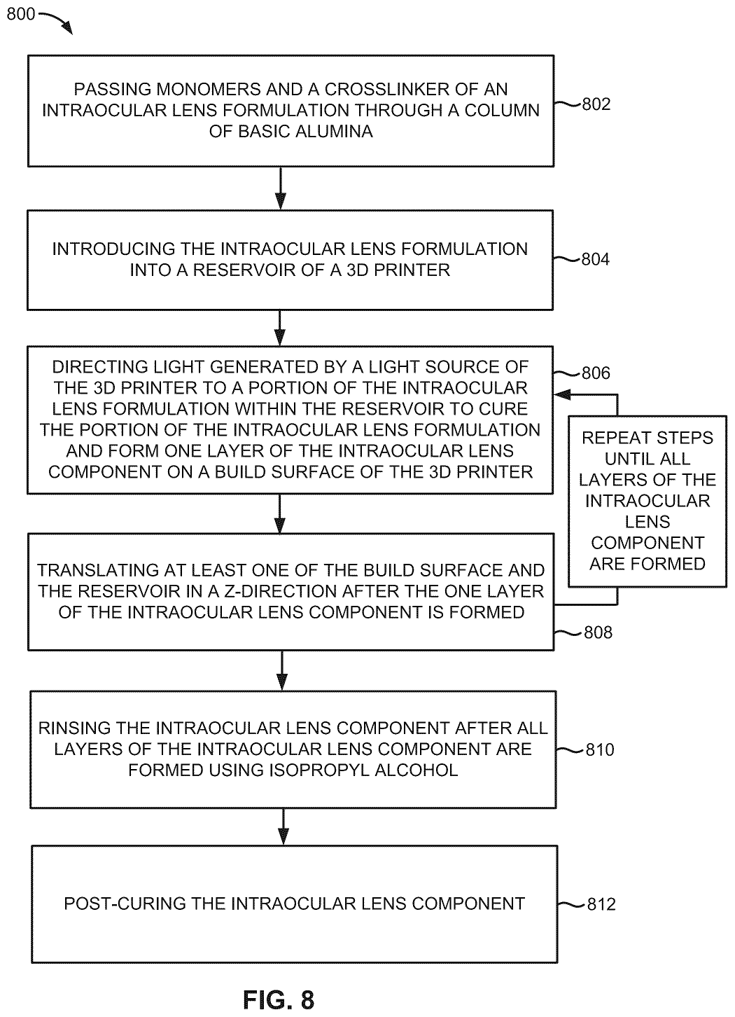

FIG. 8 is a flowchart illustrating another embodiment of a method 800 of 3D printing an intraocular lens component 101. The method 800 can comprise passing monomers and a crosslinker of an intraocular lens formulation through a basic alumina column 400 in step 802.

In some embodiments, the monomers and crosslinker of the intraocular lens formulation can be passed through the basic alumina column 400 without a solvent.

In some embodiments, the intraocular lens formulation can comprise the monomers, the crosslinker, a crosslinkable polymer comprising the plurality of monomers, and a photoinitiator. The monomers can comprise an alkyl acrylate and/or alkyl methacrylate, a phenyl acrylate or phenyl methacrylate, and, optionally, a fluoromethacrylate or a fluoroacrylate.

The method 800 can also comprise introducing the intraocular lens formulation comprising the monomers and the crosslinker that have passed through the basic alumina column 400 into a reservoir 202 of a 3D printer 200 in step 804. The intraocular lens formulation can be in liquid form when introduced into the reservoir 202.

The method 800 can also comprise directing light 214 generated by a light source 212 of the 3D printer 200 to a portion of the intraocular lens formulation within the reservoir 202 to cure the portion of the intraocular lens formulation and form one layer of the intraocular lens component 101 on a build surface 206 of the 3D printer 200 in step 806.

In some embodiments, the light 214 generated by the light source 212 can be UV light. For example, the wavelength of the UV light can be between about 365 nm and about 410 nm (e.g., 405 nm).

In some embodiments, the exposure time of the intraocular lens formulation to the light can be between about 0.1 seconds and about 10.0 seconds. The method 800 can also comprise waiting between 1 second and 900 seconds in between light exposures.

In certain embodiments, step 806 can also comprise adhering or otherwise coupling a glass plate 208 to the build surface 206 and forming the layer of the intraocular lens component 101 directly on the glass plate 208.

The method 800 can further comprise translating at least one of the build surface 206 and the reservoir 202 in a z-direction after the one layer of the intraocular lens component 101 is formed in step 808.

In some embodiments, each layer of the intraocular lens component 101 can have a thickness of between 5 μm and 50 μm. In certain embodiments, each layer of the intraocular lens component 101 can be printed in about 10 seconds. In other embodiments, each layer of the intraocular lens component 101 can be printed in between 10 seconds and 20 minutes.

The method 800 can further comprise repeating steps 806 and 808 until all layers of the intraocular lens component 101 are formed. In some embodiments, the intraocular lens component 101 can be a haptic of an intraocular lens. When the intraocular lens component is a haptic, the total print time can be between about 10 minutes and 300 minutes.

In some embodiments, the 3D printer 200 can be a photopolymerizing 3D printer capable of printing at a print resolution of between about 2 μm and 30 μm. For example, the 3D printer can be a digital light processing (DLP) 3D printer or a projection micro-stereolithography (PμSL) 3D printer.

In other embodiments, the 3D printer can be another type of stereolithography 3D printer or a two photon polymerization (2PP) 3D printer.

The method 800 can further comprise rinsing the intraocular lens component 101 after all layers of the intraocular lens component 101 are formed using isopropyl alcohol (IPA) in step 810 (e.g., 99% IPA). The method 800 can also comprise post-curing the intraocular lens component 101 after the intraocular lens component 101 is rinsed with the IPA in step 812.

In some embodiments, the intraocular lens component 101 can be post-cured using UV light. For example, the intraocular lens component 101 can be post-cured for at least 30 minutes (or between 30 minutes and 120 minutes).

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications, and journal articles) are incorporated by reference herein in their entireties except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

It is contemplated by this disclosure and it should be understood by one of ordinary skill in the art that the types of acrylic cross-linked copolymers disclosed herein can be generally copolymers of a plurality of acrylates, methacrylates, or a combination thereof and the term “acrylate” as used herein can be understood to mean acrylates, methacrylates, or a combination thereof interchangeably unless otherwise specified.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An intraocular lens formulation suitable for 3D printing, comprising:
a plurality of monomers;
a crosslinkable polymer comprising the plurality of monomers;
a crosslinker; and
a photoinitiator.

2.-22. (canceled)

23. A method of 3D printing an intraocular lens component, comprising:
(i) introducing an intraocular lens formulation into a reservoir of a 3D printer;
(ii) directing light generated by a light source of the 3D printer to a portion of the intraocular lens formulation within the reservoir to cure the portion of the intraocular lens formulation and form one layer of the intraocular lens component on a build surface of the 3D printer;
(iii) translating at least one of the build surface and the reservoir in a z-direction after the one layer of the intraocular lens component is formed; and
(iv) repeating steps (ii) and (iii) until all layers of the intraocular lens component are formed.

24. The method of claim 23, further comprising:
passing monomers of the intraocular lens formulation through a column of basic alumina; and
introducing the intraocular lens formulation comprising the monomers having passed through the column of basic alumina into the reservoir of the 3D printer.

25. The method of claim 24, wherein the monomers are passed through the column of basic alumina without a solvent.

26. The method of claim 23, wherein the light generated by the light source is ultraviolet (UV) light.

27. The method of claim 26, wherein a wavelength of the UV light is between nm and 410 nm.

28. The method of claim 23, wherein an exposure time of the intraocular lens formulation to the light is between 0.1 seconds and 10.0 seconds.

29. The method of claim 28, further comprising waiting between 1 second and seconds in between light exposures.

30. The method of claim 23, further comprising coupling a glass plate to the build surface and forming the layer of the intraocular lens component on the glass plate.

31. The method of claim 23, further comprising rinsing the intraocular lens component after all layers of the intraocular lens component are formed using isopropyl alcohol.

32. The method of claim 31, further comprising post-curing the intraocular lens component after the intraocular lens component is rinsed with the isopropyl alcohol.

33. The method of claim 32, wherein the intraocular lens component is post-cured using UV light.

34. The method of claim 32, wherein the intraocular lens component is post-cured for at least 30 minutes.

35. The method of claim 23, wherein the 3D printer is a digital light processing (DLP) 3D printer.

36. The method of claim 23, wherein the 3D printer is a projection micro-stereolithography 3D printer.

37. The method of claim 23, wherein the 3D printer has a print resolution of between 2 μm and 30 μm.

38. The method of claim 23, wherein the intraocular lens formulation is in liquid form when introduced into the reservoir of the 3D printer.

39.-43. (canceled)

44. A 3D printer for printing an intraocular lens component, comprising:
a reservoir configured to contain an intraocular lens formulation;
a build platform comprising a build surface, wherein the build surface is configured to be initially in fluid contact with the intraocular lens formulation within the reservoir, wherein at least one of the reservoir and the build platform is translatable in a z-direction;
a light source configured to generate a light; and
at least one of a mirror and a projection optic configured to direct the light generated by the light source at the intraocular lens formulation within the reservoir to cure a portion of the intraocular lens formulation and form one layer of the intraocular lens component on the build surface.

45.-54. (canceled)

55. A 3D-printed haptic, comprising:
a 3D-printed haptic body comprising a radially-outer haptic surface; and
a plurality of 3D-printing support structure remnants protruding from the radially-outer haptic surface, wherein the 3D-printing support structure remnants are formed by removing portions of 3D-printing support structures used to support a part of the 3D-printed haptic during a 3D printing process.

56.-64. (canceled)

65. A method of 3D printing a haptic of an intraocular lens, comprising:
3D printing the haptic of the intraocular lens, wherein at least part of the haptic is supported by 3D-printing support structures during the 3D printing process; and
removing portions of the 3D-printing support structures until 3D-printing support structure remnants remain along a surface of the haptic.

66.-74. (canceled)
